(19) United States
(12) Patent Application Publication
(10) Pub. No.: US 2015/0250776 A1
(43) Pub. Date: Sep. 10, 2015

(54) COMPOSITIONS AND METHODS FOR ORAL DELIVERY OF ENCAPSULATED 3-AMINOPYRIDINE-2-CARBOXALDEHYDE PARTICLES

(71) Applicant: NANOTHERAPEUTICS, INC., Alachua, FL (US)

(72) Inventors: James D. Talton, Gainesville, NC (US); Carl N. Kraus, Raleigh, NC (US)

(21) Appl. No.: 14/638,598

(22) Filed: Mar. 4, 2015

Related U.S. Application Data
(60) Provisional application No. 61/948,085, filed on Mar. 5, 2014.

(51) Int. Cl. A61K 31/44; A61K 9/50

(52) U.S. Cl. CPC A61K 31/44; A61K 9/5036

(57) ABSTRACT

The present disclosure provides compositions comprising particles, the particles comprising 3-aminopyridine-2-carboxaldehyde (3-AP) and at least one controlled-release polymer, wherein the 3-AP is encapsulated by the at least one controlled-release polymer, and pharmaceutical compositions comprising such compositions. The present disclosure also provides methods of treatment by administering an effective amount of the compositions or pharmaceutical compositions of the present disclosure, methods of making such encapsulated particle compositions, and methods of making the corresponding compositions and pharmaceutical compositions.

This application claims the benefit of U.S. Provisional Application No. 61/948,085, filed Mar. 5, 2014, which is incorporated herein by reference in its entirety.

The present disclosure relates generally to compositions comprising encapsulated particles of 3-aminopyridine-2-carboxaldehyde (3-AP), methods of preparing such encapsulated compositions, and therapeutic uses thereof. The encapsulated particle compositions described herein allow 3-AP to be administered by routes that are non-invasive to patients, such as by oral administration.

3-AP, also known as Triapine, is a potent inhibitor of ribonucleotide reductase (RR), the rate determining enzyme in the supply of deoxynucleotides (DNA building blocks) for DNA synthesis. DNA synthesis is required for cellular proliferation and DNA repair. Therefore, 3-AP has broad spectrum antitumor activity, and synergizes with antitumor drugs that target DNA. 3-AP is a very strong iron chelator and, in the body, the iron chelate may be the active species that quenches the active site tyrosyl radical required by RR for enzymatic activity. The 3-AP iron chelate is also redox active with several reports in the literature ascribing this property to some of the biological activities of 3-AP.

The mechanism by which 3-AP inhibits RR is thought to be similar to that of hydroxyurea, an agent with clinical antitumor activity against both solid tumors and hematological malignancies. However, 3-AP is 100 to 1000-fold more potent than hydroxyurea in both enzyme and tumor cell-growth inhibition assays and was shown to be active in cell lines selected for resistance to hydroxyurea. The increased activity of 3-AP compared with hydroxyurea is thought to be a consequence of its ability to chelate iron, which is essential to regenerate the tyrosyl-free radical in the M2 subunit that initiates reduction of ribonucleotides. Recently published data indicate that 3-AP may have a second mechanism contributing to its antitumor activity. A 3-AP/iron complex was shown to produce DNA damage in vitro through a redox cycling mechanism at clinically relevant concentrations. Murren et. al. (2003) conducted experiments showing preferential cytotoxic effects of 3-AP on tumor cell lines compared with a fibroblast cell line.

Oral administration of drugs, such as 3-AP, is generally preferred over intravenous administration for reasons of patient comfort and compliance. However, many drugs, including 3-AP, have poor solubility in aqueous solutions (Enyedy-2011), and are thus variably absorbed when delivered orally. Consequently, many such drugs, including 3-AP, are administered through more invasive routes, such as intravenous routes.

Several approaches for improving the oral delivery of insoluble drugs are well-known in the art. For example, poorly soluble drugs may be administered as dispersions in large amounts of fatty acids, or milled to yield nanoparticles. There has been substantial effort in the last decade to produce drug particles from 100 nanometers to a few microns because of their improved dissolution properties (especially with insoluble drugs) and ability to be absorbed more efficiently. However, each of these approaches suffers from certain drawbacks, such as inadequate stability, difficulty of manufacture, adverse interactions with the drug to be delivered, or the use of toxic amounts of permeation enhancers or enzyme inhibitors. Thus, there remains a need for compositions and methods for the non-invasive delivery of 3-AP.

Dispersible nanoparticulate compositions, as described in U.S. Pat. No. 5,145,684 (“the '684 patent”), are particles less than approximately 400 nanometers in size consisting of a poorly soluble therapeutic or diagnostic agent having absorbed onto or associated with the surface thereof a non-crosslinked surface stabilizer. The '684 patent does not describe nanoparticulate compositions of 3-AP. Methods of making nanoparticulate compositions are also described, for example, in U.S. Pat. Nos. 5,518,187 and 5,862,999, both disclosing a “Method of Grinding Pharmaceutical Substances,” and U.S. Pat. No. 5,510,118 disclosing a “Process of preparing therapeutic compositions containing nanoparticles.” Nanoparticles may be prepared by dispersing a drug substance and surface modifiers in water and wet grinding in the presence of rigid grinding media, such as silica beads or a polymeric resin. These methods require removal of the grinding media and drying as additional steps to generate a dry nanoparticle product.

Cryogenic jet-milling with nitrogen is a well-suited size reduction technique for pharmaceutical powders that may be chemically degraded by mixing in aqueous media. Using cryogenic conditions while milling easily oxidized or heat-sensitive materials controls chemical decomposition, can protect and enhance final product properties, produce finer particles/improve nanoparticle size yield, and increase the production rate. This method does not require additional steps for wet media milling described above. One method for cryogenic jet-milling with nitrogen is described in U.S. Pat. No. 8,074,906 “Process for milling and preparing powders and compositions produced thereby.”

### SUMMARY

Accordingly, one aspect of the present disclosure provides an oral composition comprising particles of 3-aminopyridine-2-carboxaldehyde (3-AP) and a controlled release polymer, wherein the 3-AP is encapsulated by the controlled-release polymer. In some embodiments, the particles are provided as a dry powder. In certain embodiments, the particles of 3-AP may have an average diameter of less than about 1 mm.

In some embodiments, the particles, the composition, or both may further comprise a second compound to enhance 3-AP dissolution, such as a surfactant.

In certain embodiments, the present disclosure provides pharmaceutical compositions comprising particles of 3-AP as a dry powder encapsulated by a controlled-release polymer and may further comprise a second compound to enhance the dissolution of 3-AP. In certain embodiments, the pharmaceutical composition may further comprise at least one excipient. In some embodiments, the dissolution of 3-AP may be enhanced by the addition of a second compound such as a surfactant.

Another aspect of the present disclosure provides a pharmaceutical composition comprising the compositions of the disclosure. In some embodiments, a pharmaceutical composition of the disclosure may provide a composition with improved aqueous dissolution comprising particles of 3-AP, starch, and sodium lauryl sulfate.

A further aspect, the present disclosure provides a method of making a composition comprising particles of 3-AP, the method comprising:
blending 3-AP together with a controlled-release polymer and, optionally, a surfactant to form a mixture;
forming coarse particles having an average diameter ranging from about 0.1 mm to about 5 mm; and
processing said coarse particles to form particles having an average diameter ranging from about 0.1 microns to about 0.1 mm.
In some embodiments, the processing step may comprise milling, grinding, or crushing.

In yet another aspect, the disclosure provides a method of inhibiting RR, such as in treating cancer or sickle cell diseases, comprising administering an effective amount of a composition or a pharmaceutical composition of the present disclosure to a patient in need thereof.

### DESCRIPTION OF THE EMBODIMENTS

### I. Particulate Delivery Systems

In some embodiments, the present disclosure provides a composition comprising a particulate delivery system or “PDS” comprising particles of an insoluble drug, such as 3-AP, and a controlled-release polymer, wherein the insoluble drug is encapsulated by the controlled-release polymer.

As used herein, the term “encapsulated” means to enclose in or as if in a capsule. Accordingly, one skilled in the art would understand that encapsulating a poorly soluble drug such as 3-AP with a controlled release polymer would produce an encapsulated particle where very little, if any, of the poorly soluble drug was exposed on the surface of the particle.

As used herein, the term “drug” encompasses the corresponding salts, hydrates, solvates, prodrugs, and complexes of a drug. Thus, a drug may be present as, e.g., a free base, a salt, a hydrate, a prodrug, a solvate (including a mixed solvate), or a complex (such as a pharmaceutically acceptable complex, and/or a complex with a polymer).

As used herein, the terms “insoluble drug,” “drug having low solubility,” and the like refer to a drug (in its neutral (i.e., uncharged) state) having a water solubility in neutral pH buffer of less than about 20 mg/ml. For example, 3-AP has poor solubility in aqueous solutions but is soluble in DMSO. Thus, as used herein, 3-AP (including 3-AP, its salts, hydrates, solvates, complexes, etc.) is an insoluble drug. In some embodiments, the present disclosure provides a pharmaceutical composition comprising a composition with improved aqueous solubility comprising a 3-AP and sodium lauryl sulfate composition of the present disclosure.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As also used herein, the articles “a,” “an” and one mean “at least one” or “one or more” of the object to which they refer, unless otherwise specified or made clear by the context in which they appear herein.

In some embodiments, an insoluble drug may be present in the compositions and PDS of the present disclosure in an amount ranging from about <10% to about 90% by weight of the PDS. For example, an insoluble drug may be present in an amount ranging from about 0.01% to about 99%, about 10% to about 90%, about 10% to about 50%, or about 10% to about 30% by weight of the PDS. In some embodiments, the insoluble drug may be present in an amount of about 25% by weight of the PDS.

In some embodiments, the controlled-release polymer may be biodegradable. In other embodiments, the controlled-release polymer may be bioerodable. In certain embodiments, the controlled-release polymer may be considered by the FDA to be generally regarded as safe (GRAS). In other embodiments, the controlled-release polymer may be, e.g., a water-soluble polymer. Exemplary polymers useful in the compositions of the disclosure include, but are not limited to, microcrystalline cellulose (e.g., Avicel®, FMC Corp., Emcocel®, Mendell Inc.), cellulose powder (Elcema, Degussa, and Solka Floc, Mendell, Inc.), crospovidone (e.g. Polyplasdone XL, International Specialty Products.), sodium starch glycolate (Explotab, Mendell Inc.), crosscarmellose sodium (e.g., Ac-Di-Sol, FMC Corp.), polyvinyl pyrrollidone, methylcellulose, hydroxypropyl methylcellulose, carboxymethyl cellulose, locust bean gum, and starch. For example, in some embodiments, the present disclosure provides a composition comprising particles of 3-AP encapsulated by a starch, such as Starch 1500, wherein the 3-AP content of the particles ranges from about 10% to about 50% by weight of the particles.

In some embodiments, a surfactant may be present in the composition and/or PDS of the present disclosure in an amount ranging from about 0.2 wt. % to about 40 wt. %, and in another embodiment from about 10 wt. % to about 30 wt. %. Further, in certain embodiments, the pharmaceutical composition comprising a composition with improved aqueous solubility may comprise 3-AP in an amount of about 25% by weight of the composition, and a surfactant, such as sodium lauryl sulfate, present in an amount of about 1% by weight of the composition.

In some embodiments, the particles, the composition, or both may further comprise a second compound to enhance 3-AP dissolution, such as a surfactant. Exemplary surfactants useful in the compositions of the disclosure include, but are not limited to magnesium stearate, calcium stearate, sodium stearate, stearic acid, sodium stearyl fumarate, hydrogenated cotton seed oil (sterotex), talc, beeswax, carnuba wax, cetyl alcohol, glyceryl stearate, glyceryl palmitate, glyceryl behenate, hydrogenated vegetable oils, and stearyl alcohol talc, corn starch, silicon dioxide, metallic stearates, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)), polyethylene glycols (e.g., Carbowaxs 3550® and 934® (Union Carbide)), polyoxyethylene stearates, and sodium lauryl sulfate. In some embodiments, the surfactant is sodium lauryl sulfate.

In some embodiments, the particles may have an average diameter of, for example, less than 5 mm, less than 3 mm, less than 2 mm, less than 600 microns, less than 500 microns, or less than 300 microns. In some embodiments, the particles may have an average diameter less than about 0.1 mm (100 microns). For example, the particles may have an average diameter of less than about 2.06 mm (corresponding to a 10 mesh sieve), less than about 1.68 mm (corresponding to a 12 mesh sieve), less than about 1.40 mm (corresponding to a 14 mesh sieve), less than about 1.20 mm (corresponding to a 16 mesh sieve), less than about 1.00 mm (corresponding to an 18 mesh sieve), less than about 0.853 mm (corresponding to a 20 mesh sieve), less than about 0.710 mm (corresponding to a 25 mesh sieve), less than about 0.599 mm (corresponding to a 30 mesh sieve), or less than about 0.500 mm (corresponding to a 35 mesh sieve). In some embodiments, the particles may have an average diameter of less than about 300 microns, and may be able to pass through a 50 mesh sieve. In certain embodiments, the particles may have an average diameter ranging from about 0.1 mm to about 5 mm, or about 0.1 microns to about 0.1 mm.

In some embodiments, a PDS according to the present disclosure may further comprise at least one additional compound, such as an additional drug. The additional drug may be chosen from, e.g., metal salts, anti-inflammatory drugs, and analgesics.

### II. Methods of Making a PDS

The present disclosure also provides a method of making a composition of the present disclosure comprising particles of an insoluble drug encapsulated by a controlled-release polymer, the method comprising:
blending 3-AP together with a controlled-release polymer and, optionally, a surfactant, to form a dry mixture;
forming coarse particles having an average diameter ranging from about 0.1 mm to about 5 mm; and
processing said coarse particles to form particles having an average diameter ranging from about 0.1 microns to about 0.1 mm.
In some embodiments, the processing step may comprise milling, grinding, or crushing. In certain embodiments, milling may be by use of a jet mill.

In certain embodiments, the particles have an average diameter ranging from about 0.1 microns to about 0.1 mm. Particulate materials, also designated as “particles”, to be produced in accordance with this disclosure include those in which small nanometer to micron size particles may be desirable. Examples may include nanoparticles and microparticle forms of pharmaceuticals, including insoluble drugs. The possibilities and combinations are numerous.

In some embodiments, a system for preparing a composition of the present disclosure may include a venturi-type nozzle or ‘Tee’ valve to introduce cryogenic gas to, for example, a jet mill. Without wishing to be bound by any particular theory, combinations of dry gases at cryogenic temperatures (generally below 0° C.) before introduction into the jet mill may be used to eliminate moisture-induced agglomeration, as well as promote brittle fracture of particles upon impaction, and has been observed to act synergistically to produce a marked improvement in the particle size reduction efficiency. Cryogenic liquids suitable for use in this method include liquid argon, liquid nitrogen, liquid helium or any other liquefied gas having a temperature sufficiently low to produce brittle fracture of particles. The cryogenic liquid may also prevent milling losses and thermal damage to the feed material that would otherwise be caused by the volatization or overheating of constituent ingredients.

In certain embodiments, a powder is placed in a temperature controlled vessel, such as a jacketed hopper or a screw-feeder, or is frozen beforehand. The cryogenic liquid and gas inputs are opened and the flow and temperature is set to the desired process conditions. The cryogenic gas input system, for example liquid nitrogen mixed with nitrogen gas as the main carrier gas in a variety of gas input setups, may be connected to a standard commercial jet mill, such as a Trost Gem-T, Trost T-15, Fluid Air Aljet, Hosikawa Alpine AS Spiral Jet Mill, Sturtevant Micronizer, or similar system. Pre-run setup of the system may include attaching a temperature probe or flowmeter, such as a TSI Model 4040 Flowmeter or similar system, at the gas input or to the top of the cyclone (in place of air relief bag), setting the carrier gas on different input pressures and documenting the gas flow and temperature measurements (CFM). The milling process may be started by turning on the powder feeder and, after passing powder through the milling region, collecting the jet-milled powder in the cup or similar receiver unit (typically particles ˜1-10 microns) or from the bag above the cyclone (particles <1 micron), depending on the exact run conditions. Particles with average diameters ranging from less than about 1 micron to about 10 microns may be produced by running the powder from the cup through the jet-mill under similar run conditions multiple times, or passes, to obtain the desired particle size.

In certain embodiments, the particles may have an average diameter ranging from about 0.1 mm (100 microns) to about 3 mm. For example, the particles may have an average diameter of less than about 2.06 mm (corresponding to a 10 mesh sieve), less than about 1.68 mm (corresponding to a 12 mesh sieve), less than about 1.40 mm (corresponding to a 14 mesh sieve), less than about 1.20 mm (corresponding to a 16 mesh sieve), less than about 1.00 mm (corresponding to an 18 mesh sieve), less than about 0.853 mm (corresponding to a 20 mesh sieve), less than about 0.710 mm (corresponding to a 25 mesh sieve), less than about 0.599 mm (corresponding to a 30 mesh sieve), or less than about 0.500 mm (corresponding to a 35 mesh sieve). In some embodiments, the particles may have an average diameter of less than about 300 microns, and may be able to pass through a 50 mesh sieve. In certain embodiments, the particles have an average diameter of about 0.6 mm or less. In some embodiments, the particles may have an average diameter ranger from about 0.1 mm to about 5 mm, or about 0.1 microns to about 0.1 mm.

In certain embodiments, the controlled-release polymer is heated prior to blending with the insoluble drug.

In some embodiments, the present disclosure provides a method of making a composition of the present disclosure comprising particles of an insoluble drug encapsulated by a controlled-release polymer using a process wherein the process is at least partially a continuous manufacturing process. The method may comprise:
blending 3-AP together with a surfactant and a controlled-release polymer and, optionally, at least one surfactant, to form a mixture;
heating said mixture to a temperature sufficient for extrusion of the mixture; extruding said mixture to form coarse particles having an average diameter ranging from about 0.1 mm to about 5 mm;
cooling said coarse particles; and
processing (e.g., by the coarse particles) said coarse particles to form particles having an average diameter less than about 0.1 mm.

In certain embodiments, the particles may have an average diameter ranging from about 0.1 mm (100 microns) to about 3 mm. For example, the particles may have an average diameter of less than about 2.06 mm (corresponding to a 10 mesh sieve), less than about 1.68 mm (corresponding to a 12 mesh sieve), less than about 1.40 mm (corresponding to a 14 mesh sieve), less than about 1.20 mm (corresponding to a 16 mesh sieve), less than about 1.00 mm (corresponding to an 18 mesh sieve), less than about 0.853 mm (corresponding to a 20 mesh sieve), less than about 0.710 mm (corresponding to a 25 mesh sieve), less than about 0.599 mm (corresponding to a 30 mesh sieve), or less than about 0.500 mm (corresponding to a 35 mesh sieve). In some embodiments, the particles may have an average diameter of less than about 300 microns, and may be able to pass through a 50 mesh sieve. In certain embodiments, the particles may have an average diameter of about 0.1 mm or less. In some embodiments, the particles may have an average diameter ranger from about 0.1 mm to about 5 mm, or about 0.1 microns to about 0.1 mm.

### III. Pharmaceutical Compositions (Final Dosage Forms)

The present disclosure further provides pharmaceutical compositions (sometimes referred to as “final dosage forms” or “FDF”) comprising a compositions according to the present disclosure.

In some embodiments, the pharmaceutical compositions may further comprise at least one excipient (such as, e.g., a controlled-release polymer, surfactant, and/or metal salt), such as a pharmaceutically acceptable excipient. Examples of pharmaceutically acceptable excipients may be, for example, those described in Remington's Pharmaceutical Sciences by E. W. Martin, and include, but are not limited to, starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. In some embodiments, the pharmaceutical compositions also contain pH buffering reagents and wetting or emulsifying agents.

In some embodiments, the pharmaceutical compositions may be formulated for oral administration. In this embodiment, the pharmaceutical composition may be in the form of, for example, tablets, capsules, or other oral dosage forms. Such oral dosage forms may be prepared by conventional means. The pharmaceutical composition can also be prepared as a liquid, for example as a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring, and sweetening agents. Alternatively, the composition can be presented as a dry product for constitution with water or another suitable vehicle.

For buccal and sublingual administration, the composition may take the form of tablets or lozenges according to conventional protocols.

The pharmaceutical composition can also be formulated for rectal administration as a suppository or retention enema, e.g., containing conventional suppository bases such as PEG, cocoa butter, or other glycerides.

In some embodiments, the pharmaceutical compositions described herein provide improved dissolution of the insoluble drug, relative to the unencapsulated poorly soluble drug, and/or to another dosage form (such as, e.g., a more invasive dosage form). For example, dissolution may be increased by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by a Vankel tablet dissolution apparatus approved by the United States Pharmacopeia.

In some embodiments, the pharmaceutical compositions described herein provide improved oral bioavailability of the poorly soluble drug, relative to the unencapsulated poorly soluble drug, and/or to another dosage form (such as, e.g., a more invasive dosage form). For example, absorption may be increased by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by, e.g., in vivo pharmacokinetic studies in a preclinical animal model or human clinical evaluation.

In some embodiments, the pharmaceutical compositions described herein are immediate-release formulations. In such embodiments, the pharmaceutical compositions provide a more rapid onset of action of the poorly soluble drug, relative to the unencapsulated poorly soluble drug, and/or to another dosage form (such as, e.g., a more invasive dosage form). For example, the onset of action may be shortened by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by, e.g., in vivo pharmacokinetic studies in a preclinical animal model or human clinical evaluation.

In some embodiments, the pharmaceutical compositions described herein are controlled-release formulations. In such embodiments, the pharmaceutical compositions described herein provide a more rapid onset of action of the insoluble drug.

In some embodiments, the pharmaceutical compositions described herein have reduced absorption variability, relative to the unencapsulated insoluble drug, and/or to another dosage form (such as, e.g., a more invasive dosage form).

In some embodiments, the pharmaceutical compositions described herein are associated with improved patient compliance, relative to another pharmaceutical composition comprising the same insoluble drug (which may be in another dosage form, such as, e.g., a more invasive dosage form).

### IV. Methods of Making Pharmaeutical Compositions

In further embodiments, the present disclosure provides a method of making a pharmaceutical composition wherein the method further comprises formulating the particles.

In certain embodiments, the particles are formulated into unit doses such as tablets or capsules.

In some embodiments wherein the pharmaceutical compositions further comprises at least one excipient, the present disclosure also provides a method of making a pharmaceutical composition wherein the method further comprises mixing the particles with at least one excipient to form a second mixture; and formulating the second mixture.

In certain embodiments, the particles are formulated into unit doses such as tablets or capsules.

### V. Methods of Treatment

The pharmaceutical compositions described herein may be useful to treat any disease or condition for which administration of a corresponding insoluble drug is desirable. For example, compositions comprising 3-AP may be useful for the treatment of iron overload or radionuclide exposure. The terms “treat,” “treatment,” and “treating” refer to (1) a reduction in severity or duration of a disease or condition, (2) the amelioration of one or more symptoms associated with a disease or condition without necessarily curing the disease or condition. In some embodiments, the method of treatment further comprises the prevention of a disease or condition. Suitable subjects include, e.g., humans and other mammals, such as, e.g., mice, rats, dogs, and non-human primates.

In yet another aspect, the disclosure provides a method of inhibiting RR, such as in treating cancer or sickle cell diseases, comprising administering an effective amount of a pharmaceutical composition of the present disclosure to a patient in need thereof.

Other embodiments of the present disclosure will be apparent to those skilled in the art from consideration of the specification and practice of the present disclosure disclosed herein. It is intended that the specification and examples be considered as exemplary only, with the true scope and spirit of the present disclosure.

What is claimed is:

1. A composition comprising particles, the particles comprising 3-aminopyridine-2-carboxaldehyde (3-AP) and at least one controlled-release polymer, wherein the 3-AP is encapsulated by the controlled-release polymer.

2. The composition according to claim 1, wherein the particles have an average diameter of less than about 1 mm.

3. The composition according to claim 1, wherein the particles have an average diameter of less than about 300 μm.

4. The composition according to claim 1, wherein the particles have an average diameter of less than about 100 μm.

5. The composition according to claim 1, wherein the particles further comprise at least one surfactant.

6. The composition according to claim 5, wherein the surfactant is chosen from magnesium stearate, calcium stearate, sodium stearate, stearic acid, sodium stearyl fumarate, hydrogenated cotton seed oil, talc, beeswax, carnuba wax, cetyl alcohol, glyceryl stearate, glyceryl palmitate, glyceryl behenate, hydrogenated vegetable oils, stearyl alcohol, talc, corn starch, silicon dioxide, metallic stearates, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, or sodium lauryl sulfate.

7. The composition according to claim 5, wherein the surfactant is sodium lauryl sulfate.

8. The composition according to claim 1, wherein the dissolution of 3-AP is improved by an amount greater than about 20% relative to non-encapsulated 3-AP.

9. The composition according to claim 1, wherein the at least one controlled-release polymer is a water-soluble polymer.

10. The composition according to claim 1, wherein the at least one controlled-release polymer is selected from microcrystalline cellulose, crospovidone, crosscarmellose sodium, polyvinyl pyrrollidone, methylcellulose, hydroxypropylmethyl cellulose, carboxymethyl cellulose, locust bean gum, and starch.

11. The composition according to claim 1, wherein the at least one controlled-release polymer is a starch.

12. The composition according to claim 1, wherein the amount of 3-AP present in the particles ranges from about 0.01% to about 99% by weight of the particles.

13. The composition according to claim 1, wherein the amount of 3-AP present in the particles ranges from about 10% to about 90% by weight of the particles.

14. The composition according to a claim 1, wherein the amount of 3-AP present in the particles ranges from about 10% to about 30% by weight of the particles.

15. A pharmaceutical composition comprising the composition according to claim 1.

16. The pharmaceutical composition according to claim 15, further comprising at least one excipient.

17. The pharmaceutical composition according to claim 16, wherein the at least one excipient is chosen from starch, sodium lauryl sulfate, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and combinations or mixtures thereof.

18. The pharmaceutical composition according to claim 15, wherein the pharmaceutical composition is formulated for oral administration.

19. The pharmaceutical composition according to claim 15, wherein the dissolution of 3-AP is improved by an amount greater than about 20% relative to non-encapsulated 3-AP.

20. A method of making a composition comprising particles, the particles comprising 3-aminopyridine-2-carboxaldehyde (3-AP) and at least one controlled-release polymer, comprising:
blending a mixture comprising the 3-AP and the at least one controlled-release polymer to form coarse particles having an average diameter ranging from about 0.1 mm to about 5 mm; and
processing said coarse particles to form particles having an average diameter less than about 0.1 microns,
wherein the 3-AP is encapsulated by the controlled-release polymer.

21. The method according to claim 20, wherein the processing step comprises milling, grinding, or crushing the coarse particles.

22. The method according to claim 20, wherein the processing step comprises jet milling the coarse particles.

23. The method according to claim 20, wherein the mixture in the blending step further comprises at least one surfactant.

24. The method according to claim 20, further comprising formulating the composition for oral administration.

25. A composition prepared by the method according to claim 20.

26. A method of treating cancer, comprising administering an effective amount of the composition according to claim 1 to a patient in need thereof.

27. A method of treating sickle cell diseases, comprising administering an effective amount of the composition according to claim 1 to a patient in need thereof.

28. A method of treating cancer, comprising administering an effective amount of the composition according to claim 15 to a patient in need thereof.

29. A method of treating sickle cell diseases, comprising administering an effective amount of the pharmaceutical composition according to claim 15 to a patient in need thereof.
